(19) United States
(12) Patent Application Publication
(10) Pub. No.: US 2006/0280786 A1
(43) Pub. Date: Dec. 14, 2006

(54) PHARMACEUTICAL FORMULATIONS FOR MINIMIZING DRUG-DRUG INTERACTIONS

(76) Inventors: Barrett E. Rabinow, Skokie, IL (US); Chyung Cook, Northbrook, IL (US); Pramod Gupta, Pittsford, NY (US)

Correspondence Address: BAXTER HEALTHCARE CORPORATION, ONE BAXTER PARKWAY, DF2-2E, DEERFIELD, IL, 60015, US

(21) Appl. No.: 11/423,491

(22) Filed: Jun. 12, 2006

Related U.S. Application Data
(60) Provisional application No. 60/690,322, filed on Jun. 14, 2005.

(51) Int. Cl. A61K 9/127; A61K 9/14

(52) U.S. Cl. 424/450; 424/489; 977/906

(57) ABSTRACT

A pharmaceutical combination for minimizing pharmacokinetic drug-drug interaction is described including a first pharmaceutical component having a particular pharmacokinetic profile in a mammal and a second pharmaceutical component formulated for parenteral administration having an altered pharmacokinetic profile different from the unaltered pharmacokinetic profile of the second pharmaceutical component, which would interfere with the pharmacokinetic profile of the first pharmaceutical component. Due to its altered pharmacokinetic profile, the second pharmaceutical component does not substantially affect the pharmacokinetic profile of the first pharmaceutical component.

### CROSS REFERENCE TO RELATED APPLICATION

This application claims the benefit of U.S. Provisional Application No. 60/690,322, filed Jun. 14, 2005.

### TECHNICAL FIELD

The present invention generally relates to the minimization of drug-drug interactions. More specifically, a pharmaceutical combination for overcoming pharmacokinetic drug-drug interactions is provided.

### BACKGROUND

A drug-drug interaction occurs when a drug which has been administered to the body induces interaction with and alters the effects of another administered drug, and when both drugs concurrently reside in the body. During a drug-drug interaction, one of the drugs exhibits an increase or decrease in therapeutic response upon interaction with the other drug.

Drug-drug interactions are categorized as either pharmacodynamic or pharmacokinetic. Pharmacodynamic drug-drug interactions generally occur when a drug enhances or decreases the effect of another drug at its action site without a change in drug concentration in the body. Pharmacodynamic interactions generally involve two or more drugs having similar or antagonizing actions, which influence a patient's sensitivity to each medication. Pharmacokinetic drug-drug interactions occur when a drug enhances or interferes with the absorption, distribution, excretion, or metabolism of another drug concurrently residing in the body. Pharmacokinetic drug-drug interactions generally result in a change in drug kinetics.

When enhancing or interfering with absorption of a drug from the gastrointestinal tract, the presence of another drug concurrently residing in the body generally increases or decreases the bioavailability of the drug by (1) altering gastrointestinal motility, gastrointestinal pH, or gastrointestinal bacterial flora; (2) forming poorly or easily absorbable chelates or complexes; (3) inducing gastrointestinal mucosal damage, or (4) initiating a binding reaction that alters the physiochemical properties of the object drug. One method for overcoming absorption complications includes staggering the respective administration times of the drugs.

When interfering with distribution of a drug, another drug concurrently residing in the body generally displaces the drug from plasma protein or tissue binding sites. More specifically, the drugs compete for protein or tissue binding sites. One of the drugs, having a higher affinity for the binding site, displaces the other drug from the binding site.

When enhancing or interfering with excretion of a drug, another drug concurrently residing in the body competes with the drug for anionic and cationic carriers, which causes changes in glomerular filtration rate, active tubular secretion, urine pH, passive tubular reabsorption, and other such renal parameters.

When enhancing or interfering with the metabolism of a drug, the presence of another drug generally alters the rate of metabolism of the drug residing in the reticuloendothelial system (RES) organs and tissue including the liver, spleen, and marrow. The RES system is alternatively referred to as the monocyte phagocytic system (MPS).

One approach for overcoming absorption, distribution, excretion, and metabolic complications is described in Swada et al., U.S. Pat. No. 6,761,895. The '895 patent describes a system for averting undesirable interaction between a drug and a concomitant drug by timed-release control of the drug or control of the site of release of the drug to the digestive tract. In purporting to overcome metabolic complications, this patent proposes a time-release control or control of the site of release from the digestive tract which is said to cause one of the drugs to reach the liver at a specific time after the concomitant drug has been absorbed in the liver. Therefore, the '895 patent proposes a system for overcoming metabolic complications without directly altering the rate of metabolism of any drug.

In view of the foregoing, it is an aspect or object of this disclosure to provide pharmaceutical combinations for overcoming pharmacokinetic drug-drug interactions including a pharmaceutical combination having a first pharmaceutical component having a particular pharmacokinetic profile in a mammal and a second pharmaceutical component formulated for parenteral administration having a modified pharmacokinetic profile. It is intended that, due to the modified formulation of the second pharmaceutical component in a modified drug delivery vehicle, the respective pharmacokinetic profiles of the respective pharmaceutical components do not substantially affect each other or at least the interaction between the respective profiles is substantially reduced compared to not formulating the second pharmaceutical component according to the invention.

Terms such as “first” and “second” are used herein to provide a convenient reference and are not intended to imply a requirement for a specific order, timing, combination or grouping of administration. The term “pharmaceutical combination” is intended to be broadly construed and intended to imply a combination of pharmaceutical components in various forms so long as each component at some point resides in a mammal concomitantly.

A pharmaceutical combination may include pharmaceutical components that are formulated to be administered separately and in different compositions. As such, a pharmaceutical component is administered to a mammal in one composition after a separate administration of another pharmaceutical component in a different composition. For example, a first pharmaceutical composition is provided in one vial (or some other administration unit), and a second pharmaceutical composition is provided in another vial (or some other administration unit), and these first and second compositions are administered separately. Such separate administration can be at different times and/or by different means of administration. Alternatively, a pharmaceutical combination may include pharmaceutical components that are formulated to be administered together. For example, a first component and second component can be administered together from a single vial (or some other administration unit) having a mixture of such components. In any of these approaches, the first and second components are understood to be administered concomitantly.

### SUMMARY OF THE INVENTION

In view of the desired goals of the invention claimed herein, pharmaceutical combinations for minimizing pharmacokinetic drug-drug interaction are provided including a first pharmaceutical component having a particular pharmacokinetic profile in a mammal and a second pharmaceutical component formulated for parenteral administration having a modified pharmacokinetic profile. Typically, the drug delivery vehicle of the second pharmaceutical component is modified and its pharmacokinetic profile is different from what it would be in an unmodified formulation. Due to the modified formulation of the second pharmaceutical component in a modified drug delivery vehicle, the respective pharmacokinetic profiles of the pharmaceutical components do not substantially affect each other, or at least the interaction between the respective profiles is substantially reduced compared to not formulating the second pharmaceutical component according to the modified formulation approach. In another aspect of this embodiment, the pharmacokinetic profile may be that of concentration variation over time. As a consequence of its formulation in a modified drug delivery vehicle, the pharmacokinetic profile of concentration variation over time for the second pharmaceutical component is different from the pharmacokinetic profile of the same component in its unmodified form. The term “modified drug delivery vehicle” in this disclosure refers to the different forms in which the second pharmaceutical component can be maintained other than a conventional liquid solution. Examples of these forms are disclosed below.

In yet another aspect of the disclosure, a method for minimizing pharmacokinetic drug-drug interaction in a mammal is provided including the steps of administering a first pharmaceutical component having a particular pharmacokinetic profile in a mammal; providing a second pharmaceutical component, the second component in a given formulation having a particular pharmacokinetic profile in the mammal, wherein the particular pharmacokinetic profile of the second pharmaceutical component in the given formulation substantially affects the pharmacokinetic profile of the first pharmaceutical component when the first and second pharmaceutical components concurrently reside within the mammal; formulating the second pharmaceutical component into a modified formulation, wherein the modified formulation alters the particular pharmacokinetic profile of the second pharmaceutical component; and administering the modified formulation of the second pharmaceutical component to the mammal parenterally. Accordingly, the altered pharmacokinetic profile of the second component does not substantially affect the pharmacokinetic profile of the first pharmaceutical component when the first pharmaceutical component and the second pharmaceutical component concurrently reside within the mammal. The second pharmaceutical component may be administered after the first pharmaceutical component and/or, the order of administration may be reversed, or the two pharmaceutical components may be administered concurrently.

In yet another embodiment, a pharmaceutical combination for minimizing drug-drug interactions in a mammal is disclosed including a first pharmaceutical component that is metabolized by a particular drug-metabolizing mechanism according to a specific metabolic timing and a second pharmaceutical component that is initially phagocytized in the RES or MPS. The second pharmaceutical component is subsequently metabolized by a similar drug-metabolizing mechanism as the first pharmaceutical component, wherein phagocytosis of the second pharmaceutical component results in a metabolic timing which is different from the metabolic timing of the second pharmaceutical component in the absence of phagocytosis. Accordingly, the pharmaceutical component formulation according to the disclosure results in different metabolic timing or timings that minimize pharmacokinetic drug-drug interaction between the first and second pharmaceutical components when the first and second pharmaceutical components concurrently reside within the mammal.

In this context, metabolic timing is defined as the concentration profile of the pharmaceutical component over time in the cells containing the drug metabolizing mechanism. In some situations, a plurality of pharmaceutical components may be present such that the total concentration of these components may exceed the capacity of (i.e. saturate) the drug-metabolizing enzymes, inhibiting metabolism of the components. In one aspect of this embodiment, the formulation of one or more of the components in a modified drug delivery vehicle reduces the sum of the concentrations of the components, so as to reduce the likelihood that the enzyme(s) will become saturated.

In yet another aspect, a method for minimizing pharmacokinetic drug-drug interaction in a mammal is provided including the steps of administering to the mammal a first pharmaceutical component that is metabolized by a particular drug-metabolizing mechanism according to a specific metabolic timing; providing a second pharmaceutical component, the second component in a given formulation, when administered to the mammal, is metabolized by a similar drug-metabolizing mechanism and according to a similar metabolic timing as the first pharmaceutical component; modifying the formulation of the second pharmaceutical component, wherein the modified formulation, when administered to the mammal, causes the second pharmaceutical component to be phagocytized in the RES or MPS; and administering the modified formulation of the second pharmaceutical component to the mammal parenterally. In this embodiment, phagocytosis of the modified formulation of the second pharmaceutical component results in a metabolic timing which is different from the metabolic timing of what it would be in the absence of phagocytosis, such that the common metabolizing enzymes of the two pharmaceutical components are not saturated. Accordingly, the different metabolic timings minimize pharmacokinetic drug-drug interaction between the first pharmaceutical component and the second pharmaceutical component when the first pharmaceutical component and the second pharmaceutical component concurrently reside within the mammal. Alternatively, the first pharmaceutical component may be administered after the second pharmaceutical component.

It should be understood that the present invention includes a number of different aspects or features which may have utility alone and/or in combination with other aspects or features. Accordingly, this summary is not an exhaustive identification of each such aspect or feature that is now or may hereafter be claimed, but represents an overview of certain aspects of the present invention to assist in understanding the more detailed description that follows. The scope of the invention is not limited to the specific embodiments described below, but is set forth in the claims now or hereafter filed.

### BRIEF DESCRIPTION OF THE SEVERAL VIEWS OF THE DRAWINGS

Throughout this description, reference has been and will be made to the accompanying views of the drawing wherein like subject matter has like reference numerals, and wherein:

FIG. 1 is a diagrammatic representation of a method of producing a nanoparticulate pharmaceutical component having a modulated pharmacokinetic profile in accordance with an embodiment of the present disclosure;

FIG. 2 is a diagrammatic representation of another method of producing a nanoparticulate pharmaceutical component having a modulated pharmacokinetic profile in accordance with an embodiment of the present disclosure; and

FIG. 3 is a graphical representation that illustrates the intravenous pharmacokinetic profiles of concentration variation over time for itraconazole in nanosuspension form as compared to a solution formulation of itraconazole.

### DETAILED DESCRIPTION OF THE MULTIPLE EMBODIMENTS

Traditional pharmaceutical combinations may comprise a number of pharmaceutical components, which may exhibit drug-drug interaction. In traditional drug delivery, two or more pharmaceutical components may be metabolized by similar drug metabolizing mechanisms, for example via similar species of drug-metabolizing enzymes. Therefore, if they concurrently reside within a mammal, these pharmaceutical components will compete for the same species of drug-metabolizing enzymes, thereby causing undesirable drug-drug interaction.

For example, it is often found that pharmaceutical components are metabolized by the CYP enzyme system (e.g., the cytokine P-450 enzymes located in the liver microsomes). There are a limited number of enzyme molecules comprising this system; therefore, generally the capacity of any one of the enzyme molecules is limited. If drugs which reside concurrently are metabolized by the same enzyme molecules, one drug will interfere with and affect the other drug's plasma concentration. This occurs because the enzymes are saturable, and do not have infinite capacity to metabolize all compounds simultaneously.

Serious side effects have resulted from the coadministration of drugs which interfere with the metabolism of other drugs. For example, the coadministration of ketoconazole and terfenadine has caused potentially life-threatening ventricular arrhythmias. Furthermore, the coadministration of sorivudine and flurouracil has resulted in fatal toxicity. In such cases, where a drug causes a reduced metabolism of another drug in the liver microsomes, excessively high plasma concentration of that first drug levels result in high levels of toxicity.

In one aspect of this disclosure, a pharmaceutical combination is provided having a first pharmaceutical component having a particular pharmacokinetic profile in a mammal and a second pharmaceutical component in a modified formulation. Due to its formulation in a modified drug delivery vehicle, the pharmacokinetic profile of the second pharmaceutical component is changed compared to its unformulated state, and the modified second pharmaceutical component does not substantially affect the pharmacokinetic profile of the first pharmaceutical component or the effects are reduced.

In another aspect of this disclosure, an individual receives the same total effective dose of the second pharmaceutical component in the formulated state as would occur in the unformulated state, but as a consequence of the formulation the plasma concentration levels of the second pharmaceutical component are reduced compared to those in the unformulated state. The reduced plasma concentration levels of the second pharmaceutical component in the formulated state result in a reduction of the inhibition of the drug metabolizing system compared to the unformulated state because there is less competition by the second pharmaceutical component with the first pharmaceutical component for the drug-metabolizing enzymes. The second component is reformulated such that the plasma concentration levels are reduced relative to the unmodified state, so as to cause less inhibition of the common enzyme system. This is accomplished by prolonging the plasma half-life for the reformulated pharmaceutical component relative to its unformulated state. Therefore, in accordance with an aspect of the invention, a method is provided wherein the unformulated second pharmaceutical component exhibits a given average plasma concentration over a certain period of time when administered to a mammal at a selected dose, and wherein the reformulated second pharmaceutical component exhibits a lower average plasma concentration over a longer period of time when administered to the mammal at the same selected dose.

In yet another embodiment, a second pharmaceutical component is provided which is metabolized by a similar species of drug-metabolizing enzyme as the first pharmaceutical component. In order to minimize drug-drug interaction, the second pharmaceutical component is formulated for parenteral administration such that it is initially phagocytized by the RES or MPS. More specifically, upon parenteral administration, the second pharmaceutical component is generally not readily soluble in the blood, and is recognized as being a foreign body requiring elimination from systemic circulation. Accordingly, the second pharmaceutical component is sequestered by fixed macrophages in the RES or MPS via phagocytosis. The organs or tissues generally associated with phagocytosis are the liver, spleen, and marrow. Enveloped in the fixed macrophages, the pharmaceutical component dissolves therefrom, enabling it to migrate out of the phagolysozomes and then to the extracellular milieu. In this context, dissolution refers to the process where the phagolysozome changes the form of the pharmaceutical component such that it is able to egress out of the MPS to the extracellular milieu. Although not wishing to be bound by theory, this egression may involve a passive diffusion of a solubilized molecule of the pharmaceutical component through a biological membrane or removal through the exocytotic pathway. Alternatively, the macrophages containing the second pharmaceutical component may die and other macrophages may scavenge the second pharmaceutical component and repeat the process. Alternatively, other mechanisms may also operate.

In this way, the phagocytosis, dissolution and transport from the fixed macrophages causes the second pharmaceutical component to have a metabolic timing which is different from the metabolic timing of the first pharmaceutical component. Accordingly, the different metabolic timings minimize pharmacokinetic drug-drug interaction between the first and second pharmaceutical components when the first and second pharmaceutical components concurrently reside within the mammal.

While the invention is susceptible of embodiment in many different forms and in various combinations, particular focus will be on the multiple embodiments of the invention described herein with the understanding that such embodiments are to be considered exemplifications of the principles of the invention and are not intended to limit the broad aspect of the invention.

For example, in accordance with the teachings of the present disclosure, the subject pharmaceutical combination generally includes a first pharmaceutical component having a particular pharmacokinetic profile and second pharmaceutical component present in a formulation that alters the pharmacokinetic profile of the second pharmaceutical component compared to the unformulated state.

The first pharmaceutical component may be administered by a number of routes including, but not limited to, parenteral, oral, buccal, periodontal, rectal, nasal, pulmonary, topical, transdermal, intravenous, intramuscular, subcutaneous, intradermal, intraoccular, intracerebral, intralymphatic, pulmonary, intraarcticular, intrathecal and intraperitoneal administration. Moreover, a liquid dispersion form of the submicron particles of the pharmaceutical component may be prepared including, but not limited to, injectable formulations, solutions, delayed release formulations, controlled release formulations, extended release formulations, pulsatile release formulations and immediate release formulations.

A solid dosage of the first pharmaceutical component may further be prepared in the form of tablets, coated tablets, capsules, ampoules, suppositories, lyophilized formulations, delayed release formulations, controlled release formulations, extended release formulations, pulsatile release formulations, immediate release and controlled release formulations administered through patches, powder preparations which can be inhaled, suspensions, creams, ointments, and other such solid dosage administration means.

The second pharmaceutical components having a modulated pharmacokinetic profile are generally poorly soluble drugs having an aqueous solubility of not greater than about 10 mg/ml. Such drugs further provide challenges to delivering them in an injectable form such as through parenteral administration. In order to facilitate their delivery, poorly soluble or insoluble drugs and/or their drug delivery vehicles have been modified under the approaches as discussed herein.

Methods for modification of the drug itself in an attempt to render it more suitable for the administration avenue chosen include altering the formulation or molecular structure of the drug. Methods for drug delivery vehicle modification of poorly soluble or insoluble drugs include the use of salt formation, solid carrier systems, co-solvent/solubilization, micellization, lipid vesicle, oil-water partitioning, liposomes, microemulsions, emulsions, and complexation.

Yet another method for vehicle modification includes nanoparticles in a solid particle suspension. Drugs that are insoluble in water can provide the significant benefit of stability when formulated as a stable suspension of sub-micron particles in an aqueous medium. Accurate control of particle size is essential for safe and efficacious use of these formulations. Particles should not be greater than seven microns in diameter to safely pass through capillaries without causing emboli (Allen et al., 1987; Davis and Taube, 1978; Schroeder et al., 1978; Yokel et al., 1981).

Accordingly, in order to minimize drug-drug interactions among a number of pharmaceutical components within a pharmaceutical combination in accordance with the teachings of the present disclosure, the pharmaceutical combination may include at least one pharmaceutical component having a modulated pharmacokinetic profile achieved through drug delivery vehicle modification of the component. Modulating the pharmacokinetic profile through nanoparticles, nanosuspensions, microemulsions, emulsions, micelles, and liposomes are explained in detail hereinafter for exemplary purposes only. Further, nanoparticles, nanosuspensions, emulsions, micelles, and liposomes each have different rates of phagocytosis and dissolution within the RES or MPS. Accordingly, the rate of dissolution and release by the macrophages within the RES or MPS and, in effect, the drug-drug interaction between pharmaceutical components within a pharmaceutical combination may be controlled using varying methods of delivery.

Nanoparticles

In order to minimize drug-drug interactions among a number of pharmaceutical components within a pharmaceutical combination in accordance with the teachings of the present disclosure, the pharmaceutical combination may include at least one pharmaceutical component having a modulated pharmacokinetic profile achieved through forming a nanoparticle of the component.

Nanoparticles of poorly soluble pharmaceutical components, in accordance with the teachings of the present disclosure, may be prepared in a number of different ways. These methods of preparing nanoparticles include, but are not limited to, preparation of solvent-free suspension, replacement of excipients, lyophilization, emulsion precipitation, solvent anti-solvent precipitation, phase inversion precipitation, pH shift precipitation, infusion precipitation, temperature shift precipitation, solvent evaporation precipitation, reaction precipitation, compressed fluid precipitation, mechanical grinding of an active agent, or any other method for producing suspensions of poorly soluble submicron particles as described in U.S. Pat. Nos. 6,607,784; 5,560,932; 5,662,883; 5,665,331; 5,145,684; 5,510,118; 5,518,187; 5,534,270; 5,718,388; and 5,862,999; in U.S. Patent Application Publication Nos. 2005/0037083; 2004/0245662; 2004/0164194; 2004/0173696; 2004-0022862; 2003/0100568; 2003/0096013; 2003/0077329; 2003/0072807; 2003/0059472; 2003/0044433; 2003/0031719; 2002/176935; 2002/0127278; and 2002/0168402, and in commonly assigned and co-pending U.S. Patent Application Ser. Nos. 60/258,160 and 60/347,548. These patents, patent publications, patent applications and all other patents, patent publications, patent applications, articles, or other references mentioned herein are hereby incorporated herein by reference and made a part hereof.

I. Nanosuspensions

One approach for delivering a poorly soluble drug using a solid particle suspension is providing what are commonly referred to as nanosuspensions. Nanosuspensions generally include aqueous suspensions of nanoparticles of relatively insoluble drug agents. Nanoparticles also generally are coated with one or more surfactants or other excipients of a particulate in order to prevent agglomeration or flocculation of the nanoparticles. Surfactants generally used for such coating preferably include, but are not limited to, ionic surfactants, nonionic surfactants, zwitterionic surfactants, phospholipids, biologically derived surfactants or amino acids and their derivatives.

One approach to preparing a nanosuspension is described in Kipp et al. U.S. Pat. No. 6,607,784. The '784 patent discloses a method for preparing submicron sized particles of an organic compound, wherein the solubility of the organic compound is greater in a water-miscible selected solvent than in another solvent which is aqueous. The process described in the '784 patent generally includes the steps of (i) dissolving the organic compound in the water-miscible selected solvent to form a solution, (ii) mixing the solution with another solvent to define a pre-suspension; and (iii) adding energy to the pre-suspension to form particles which can be of submicron size. The particles range in particle size from about 10 nm to about 10 microns, but preferably from about 100 nm to about 1000 nm or 1 micron. Often, the average effective particle size can range between about 400 nm or below, extending into low micron size, and typically no greater than about 2 microns.

The multiple nanosuspension embodiments as described in detail herein refer and/or relate to the preparation of nanosuspensions including nanoparticles of poorly soluble pharmaceuticals using an energy addition method. The entire class of poorly soluble pharmaceutical components, analogs of pharmaceutical components, and other equivalent methods for preparing nanosuspensions may be produced in submicron form without deviating from the spirit of the present invention. Energy addition methods and equipment for preparing particle suspensions of the present invention are disclosed in the commonly assigned '784 patent. A general procedure for preparing the suspension useful in the practice of this nanosuspension aspect of the invention follows.

The processes of this type can be separated into three general categories. Each of the categories of processes share the steps of: (i) dissolving the organic compound in a water-miscible selected solvent to form a solution, (ii) mixing the solution with another solvent to define a pre-suspension; and (iii) adding energy to the pre-suspension to form particles having an average effective particle size as discussed herein.

A. First Process Category for Nanosuspension Preparation

The methods of the first process category for nanosuspension preparations generally include dissolving a pharmaceutical component to have the modulated pharmacokinetic profile in a water miscible selected solvent to form a solution. This resulting solution including the pharmaceutical component can be in an amorphous form, a semi-crystalline form or a super-cooled liquid form. The selected solvent according to this nanosuspension aspect is a solvent or mixture of solvents in which the organic compound of interest is relatively soluble and which is miscible with the other solvent. Such solvents include, but are not limited to, water-miscible protic compounds, in which a hydrogen atom in the molecule is bound to an electronegative atom such as oxygen, nitrogen, or other Group V A, Group VI A and Group VII elements A in the Periodic Table of Elements. Examples of such solvents include, but are not limited to, alcohols, amines (primary or secondary), oximes, hydroxamic acids, carboxylic acids, sulfonic acids, phosphonic acids, phosphoric acids, amides and ureas.

Other examples of the selected solvent also include aprotic organic solvents. Some of these aprotic solvents can form hydrogen bonds with water, but can only act as proton acceptors because they lack effective proton donating groups. One class of aprotic solvents is a dipolar aprotic solvent, as defined by the International Union of Pure and Applied Chemistry (IUPAC Compendium of Chemical Terminology, 2nd Ed., 1997):
A solvent with a comparatively high relative permittivity (or dielectric constant), greater than ca. 15, and a sizable permanent dipole moment, that cannot donate suitably labile hydrogen atoms to form strong hydrogen bonds, e.g. dimethyl sulfoxide.

Dipolar aprotic solvents can be selected from the group consisting of: amides (fully substituted, with nitrogen lacking attached hydrogen atoms), ureas (fully substituted, with no hydrogen atoms attached to nitrogen), ethers, cyclic ethers, nitriles, ketones, sulfones, sulfoxides, fully substituted phosphates, phosphonate esters, phosphoramides, nitro compounds, and the like. Dimethylsulfoxide (DMSO), N-methyl-2-pyrrolidinone (NMP), 2-pyrrolidinone, 1,3-dimethylimidazolidinone (DMI), dimethylacetamide (DMA), dimethylformamide (DMF), dioxane, acetone, tetrahydrofuran (THF), tetramethylenesulfone (sulfolane), acetonitrile, and hexamethylphosphoramide (HMPA), nitromethane, among others, are members of this class.

Solvents also may be chosen that are generally water-immiscible, but have sufficient water solubility at low volumes (not greater than 10%) to act as a water-miscible first solvent at these reduced volumes. Examples include aromatic hydrocarbons, alkenes, alkanes, and halogenated aromatics, halogenated alkenes and halogenated alkanes. Aromatics include, but are not limited to, benzene (substituted or unsubstituted), and monocyclic or polycyclic arenes. Examples of substituted benzenes include, but are not limited to, xylenes (ortho, meta, or para), and toluene. Examples of alkanes include but are not limited to hexane, neopentane, heptane, isooctane, and cyclohexane. Examples of halogenated aromatics include, but are not restricted to, chlorobenzene, bromobenzene, and chlorotoluene. Examples of halogenated alkanes and alkenes include, but are not restricted to, trichloroethane, methylene chloride, ethylenedichloride (EDC), and the like.

Examples of the all of the above solvent classes include but are not limited to: N-methyl-2-pyrrolidinone (N-methyl-2-pyrrolidone), 2—pyrrolidinone (2-pyrrolidone), 1,3-dimethyl-2-imidazolidinone (DMI), dimethylsulfoxide, dimethylacetamide, carboxylic acids (such as acetic acid and lactic acid), aliphatic alcohols (such as methanol, ethanol, isopropanol, 3-pentanol, and n-propanol), benzyl alcohol, glycerol, butylene glycol (1,2-butanediol, 1,3-butanediol, 1,4-butanediol, and 2,3-butanediol), ethylene glycol, propylene glycol, mono- and diacylated glycerides, dimethyl isosorbide, acetone, dimethylsulfone, dimethylformamide, 1,4-dioxane, tetramethylenesulfone (sulfolane), acetonitrile, nitromethane, tetramethylurea, hexamethylphosphoramide (HMPA), tetrahydrofuran (THF), diethylether, tert-butylmethyl ether (TBME), aromatic hydrocarbons, alkenes, alkanes, halogenated aromatics, halogenated alkenes, halogenated alkanes, xylene, toluene, benzene, substituted benzene, ethyl acetate, methyl acetate, butyl acetate, chlorobenzene, bromobenzene, chlorotoluene, trichloroethane, methylene chloride, ethylenedichloride (EDC), hexane, neopentane, heptane, isooctane, cyclohexane, polyethylene glycol (PEG), PEG esters, PEG-4, PEG-8, PEG-9, PEG-12, PEG-14, PEG-16, PEG-120, PEG-75, PEG-150, polyethylene glycol esters, PEG-4 dilaurate, PEG-20 dilaurate, PEG-6 isostearate, PEG-8 palmitostearate, PEG-150 palmitostearate, polyethylene glycol sorbitans, PEG-20 sorbitan isostearate, polyethylene glycol monoalkyl ethers, PEG-3 dimethyl ether, PEG-4 dimethyl ether, polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, and glycofurol (tetrahydrofurfuryl alcohol polyethylene glycol ether).

A preferred selected solvent is N-methyl-2-pyrrolidinone (NMP). Another preferred selected solvent is lactic acid.

B. Second Process Category for Nanosuspension Preparation

The second process category for nanosuspension preparation involves mixing the solution of the first process category with another solvent to precipitate the pharmaceutical component to define a pre-suspension. In this process category, the pre-suspension of the pharmaceutical component becomes crystalline in form. After the first two process steps, the pharmaceutical component in the pre-suspension is in a friable form having an average effective particle size (e.g., such as slender needles and thin plates), thereby ensuring the particles of the presuspension are in a friable state, a state wherein the organic compound is fragile. Compounds in the friable state can also more easily and more quickly be prepared into a particle within the desired size ranges when compared to processing an organic compound where approaches have not been taken to render it in a friable form.

This other solvent used in the second process category is generally an aqueous solvent. This aqueous solvent may be water by itself. This solvent may also contain buffers, salts, surfactant(s), water-soluble polymers, and combinations of these excipients.

C. Third Process Category for Nanosuspension Preparation

The third process category for nanosuspension preparation involves adding energy to the pre-suspension which results in a breaking up and coating of the friable particles. The energy-addition step can be carried out in any fashion wherein the pre-suspension is exposed to cavitation, shearing or impact forces. In one preferred form of the invention, the energy-addition step is an annealing step. Annealing is defined in this disclosure as the process of converting matter that is thermodynamically unstable into a more stable form by single or repeated application of energy (direct heat or mechanical stress), followed by thermal relaxation. This lowering of energy may be achieved by conversion of the solid form from a less ordered to form a more ordered lattice structure. Alternatively, this stabilization may occur by a reordering of the surfactant molecules at the solid-liquid interface.

1. Method A for Nanosuspension Preparation

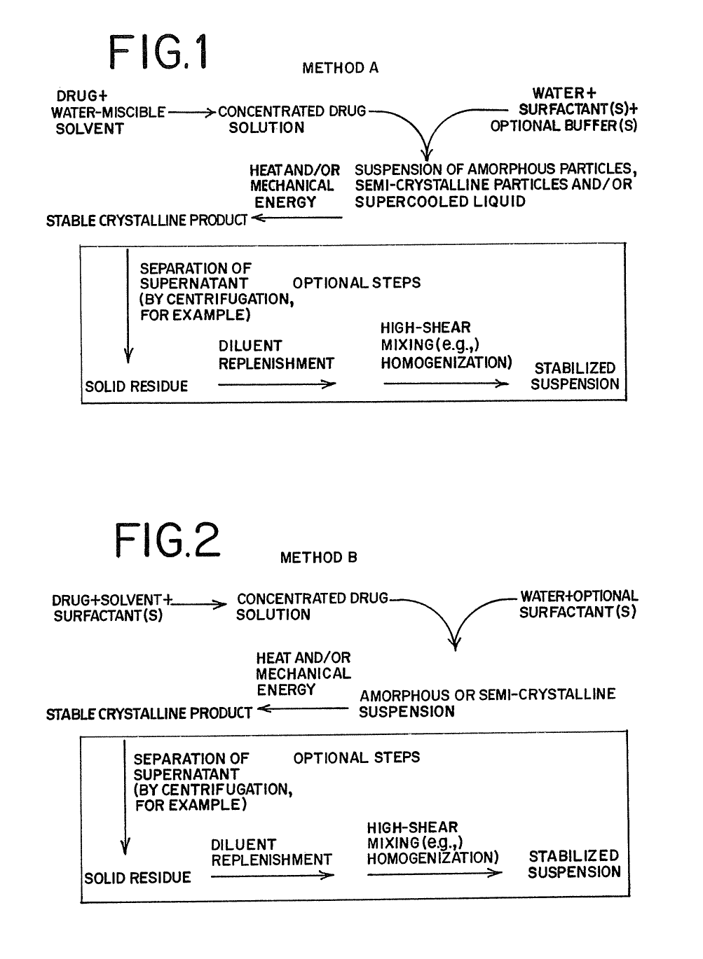

As illustrated in FIG. 1, in Method A for nanosuspension preparation, the pharmaceutical component to have the modulated pharmacokinetic profile is first dissolved in a selected solvent to create a first solution. The first solution may be heated from about 30° C. to about 100° C. to ensure total dissolution of the pharmaceutical component in the selected solvent.

Another aqueous solution is provided with one or more surfactants added thereto. The surfactant or surfactants can be selected from an ionic surfactant, a nonionic surfactant, a cationic surfactant, a zwitterionic surfactant, a phospholipid, or a biologically derived surfactant. Suitable surfactants for coating the particles in the present invention can be selected from ionic surfactants, nonionic surfactants, zwitterionic surfactants, phospholipids, biologically derived surfactants or amino acids and their derivatives. Ionic surfactants can be anionic or cationic. The surfactants are present in the components in an amount of from about 0.01% to 10% w/v, and preferably from about 0.05% to about 5% w/v.

Suitable anionic surfactants include but are not limited to: alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, (e.g. calcium stearate), phosphates, sodium dodecylsulfate, carboxymethylcellulose calcium, carboxymethylcellulose sodium, dioctylsulfosuccinate, dialkylesters of sodium sulfosuccinic acid, sodium lauryl sulfate and phospholipids.

Suitable cationic surfactants include but are not limited to: quaternary ammonium compounds, benzalkonium chloride, cetyltrimethylammonium bromide, chitosans, lauryldimethylbenzylammonium chloride, acyl carnitine hydrochlorides, alkyl pyridinium halides, cetyl pyridinium chloride, cationic lipids, polymethylmethacrylate trimethylanmonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, “POLYQUAT 10” (a mixture of polymeric quartenary ammonium compounds), tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, “MIRAPOL,” (polyquaternium-2) “ALKAQUAT”, alkyl pyridinium salts, amines, amine salts, imide azolinium salts, protonated quaternary acrylamides, methylated quaternary polymers, and cationic guar gum, benzalkonium chloride, dodecyl trimethyl ammonium bromide, triethanolamine, and poloxamines.

Suitable nonionic surfactants include but are not limited to: polyoxyethylene fatty alcohol ethers, polyoxyethylene sorbitan fatty acid esters, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylinethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, C₁₈H₃₇CH₂C(O)N(CH₃)CH₂(CHOH)₄(CH₂OH)₂, p-isononylphenoxypoly(glycidol), decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopy-ranoside, n-heptyl-β-D-thioglucoside, n-hexyl-β-D-glucopyranoside; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof.

Suitable biologically derived surfactants include, but are not limited to: lipoproteins, gelatin, casein, lysozyme, albumin, casein, heparin, hirudin, or other proteins. The preferred surfactant is a combination of an ionic surfactant (e.g., deoxycholic acid) and a nonionic surfactant (e.g., polyoxyethylene-polypropylene block copolymers such as Poloxamer 188). Another preferred surfactant is a combination of phospholipids such as Lipoid E80 and DSPE-PEG₂₀₀₀.

It may also be desirable to add a pH adjusting agent to the aqueous surfactant solution such as sodium hydroxide, hydrochloric acid, an amino acid such as glycine, tris buffer or citrate, acetate, lactate, meglumine, or the like. The aqueous surfactant solution preferably has a pH within the range of from about 2 to about 12. Suitable pH adjusting agents include, but are not limited to, sodium hydroxide, hydrochloric acid, tris buffer, mono-, di-, tricarboxylic acids and their salts, citrate buffer, phosphate, glycerol-1-phosphate, glycercol-2-phosphate, acetate, lactate, tris(hydroxymethyl)aminomethane, aminosaccharides, mono-, di- and trialkylated amines, meglumine (N-methylglucosamine), and amino acids.

The aqueous surfactant solution may additionally include an osmotic pressure adjusting agent, such as but not limited to glycerin, a monosaccharide such as dextrose, a disaccharide such as sucrose, trehalose and maltose, a trisaccharide such as raffinose, and sugar alcohols such as mannitol and sorbitol.

The aqueous surfactant solution of the particle suspension component may further be removed to form dry particles. The method to remove the aqueous medium can be any method known in the art. One example is evaporation. Another example is freeze-drying or lyophilization. The dry particles may then be formulated into any acceptable physical form including, but not limited to, solutions, tablets, capsules, suspensions, creams, lotions, emulsions, aerosols, powders, incorporation into reservoir or matrix devices for sustained release (such as implants or transdermal patches), and the like. The aqueous suspension of the present invention may also be frozen to improve stability upon storage. Freezing of an aqueous suspension to improve stability is disclosed in the commonly assigned and co-pending U.S Patent Application Publication No. 2003/0077329.

The pharmaceutical component solution and aqueous surfactant solution are then combined. Preferably, the pharmaceutical component solution is added to the aqueous surfactant solution at a controlled rate. The addition rate is dependent on the batch size, and precipitation kinetics for the pharmaceutical component. During the addition, the solutions should be under constant agitation. It has been observed using light microscopy that amorphous particles, semi-crystalline solids, or a super-cooled liquid are formed to create a pre-suspension. The method further includes the step of subjecting the pre-suspension to an annealing step to convert the amorphous particles, super-cooled liquid or semi-crystalline solid to a crystalline more stable solid state. The resulting particles will have an average effective particle size as measured by dynamic light scattering methods (e.g., photocorrelation spectroscopy, laser diffraction, low-angle laser light scattering (LALLS), medium-angle laser light scattering (MALLS)), light obscuration methods (Coulter method, for example), rheology, or microscopy (light or electron) within the ranges set forth above.

The energy-addition step for producing nanosuspensions involves adding energy through sonication, homogenization, counter-current flow homogenization (e.g., the Mini DeBEE 2000 homogenizer, available from BEE Incorporated, NC, in which a jet of fluid is directed along a first path, and a structure is interposed in the first path to cause the fluid to be redirected in a controlled flow path along a new path to cause emulsification or mixing of the fluid), microfluidization, or other methods of providing impact, shear or cavitation forces, including other homogenization approaches. The sample may be cooled or heated during this stage. In one preferred form of this aspect the invention, the annealing step is effected by homogenization. In another preferred form of this aspect of the invention, the annealing may be accomplished by ultrasonication. In yet another preferred form, the annealing may be accomplished by use of an emulsification apparatus as described in U.S. Pat. No. 5,720,551.

Depending upon the rate of annealing, it may be desirable to adjust the temperature of the processed sample to within the range of from approximately 0° C. to 30° C. Alternatively, in order to effect a desired phase change in the processed solid, it may also be necessary to adjust the temperature of the pre-suspension to a temperature within the range of from about −30° C. to about 100° C. during the annealing step.

2. Method B for Nanosuspension Preparation

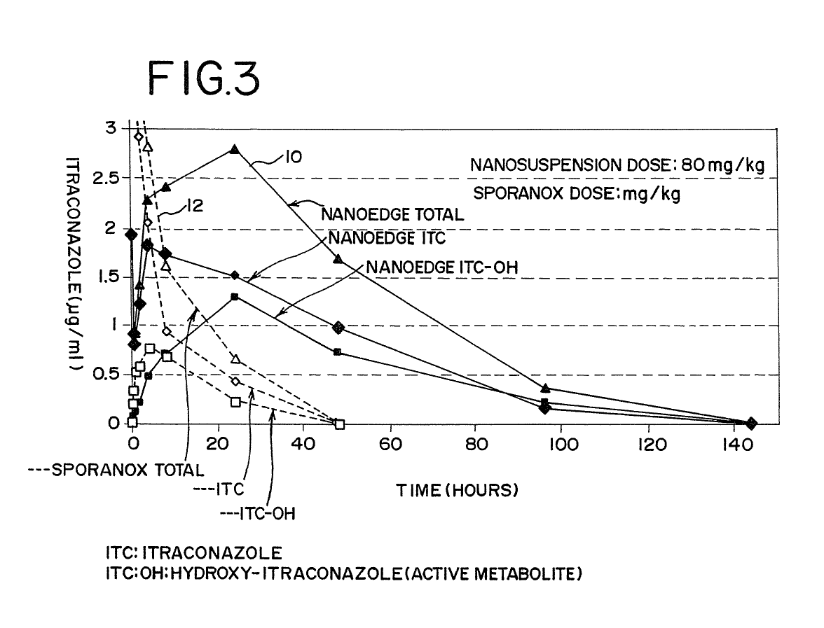

As illustrated in FIG. 2, Method B for preparing nanosuspensions includes the addition of a surfactant or combination of surfactants to the first solution. The surfactants may be selected from ionic surfactants, nonionic surfactants, cationic surfactants, zwitterionic surfactants, phospholipids, or biologically derived as set forth above. A drug suspension resulting from application of the processes described in this invention may be administered directly as an injectable solution, provided that an appropriate means for solution sterilization is applied. Method B for preparing nanosuspensions also typically includes further procedures such as preparation of solvent-free suspension, replacement of excipients, lyophilization, solvent anti-solvent precipitation, phase inversion precipitation, pH shift precipitation, infusion precipitation, temperature shift precipitation, solvent evaporation precipitation, reaction precipitation, and compressed fluid precipitation.

Preparation of Solvent-Free Suspension

Nanosuspension preparation optionally can include a solvent-free suspension, which may be produced by solvent removal after precipitation. This can be accomplished by centrifugation, dialysis, diafiltration, force-field fractionation, high-pressure filtration or other separation techniques well known in the art, such as the following. Complete removal of lactic acid or N-methyl-2-pyrrolidinone, for example, is typically carried out by one to three successive centrifugation runs; after each centrifugation the supernatant is decanted and discarded. A fresh volume of the suspension vehicle without the organic solvent is added to the remaining solids and the mixture was dispersed by homogenization. It will be recognized by others skilled in the art that other high-shear mixing techniques could be applied in this reconstitution step.

Replacement of Excipients

Furthermore, any undesired excipients such as surfactants may be replaced by a more desirable excipient by use of the separation methods described in the above paragraph. The solvent and first excipient may be discarded with the supernatant after centrifugation or filtration. A fresh volume of the suspension vehicle without the solvent and without the first excipient may then be added. Alternatively, a new surfactant may be added. For example, a suspension consisting of drug, N-methyl-2-pyrrolidinone (solvent), Poloxamer 188 (first excipient), sodium deoxycholate, glycerol and water may be replaced with phospholipids (new surfactant), glycerol and water after centrifugation and removal of the supernatant.

Lyophilization

The suspension may be dried by lyophilization (freeze-drying) to form a lyophilized suspension for reconstitution into a suspension suitable for administration. For the purpose of preparing a stabilized, dry solid, bulking agent such as mannitol, sorbitol, sucrose, starch, lactose, trehalose or raffinose may be added prior to lyophilization. The suspension may be lyophilized using any applicable program for lyophilization, for example: loading at +25° C.; cooling down to −45° C. in 1 hour; holding time at −45° C. for 3.5 hours; mean drying for 33 hours with continual increase of temperature to +15° C. at a pressure of 0.4 mbar; final drying for 10 hours at +20° C. at a pressure of 0.03 mbar; and cryo protectant: mannitol.

In addition to the microprecipitation methods described above, any other known precipitation methods for preparing particles of active agent (and more preferably, nanoparticles) in the art can be used in conjunction with this Method B nanosuspension aspect of the present invention. The following is a description of examples of other precipitation methods. The examples are for illustration purposes, and are not intended to limit the scope of the present invention.

Solvent Anti-Solvent Precipitation

Another precipitation technique is solvent anti-solvent precipitation. Suitable solvent anti-solvent precipitation technique is described in U.S. Pat. Nos. 5,118,528 and 5,100,591. The process includes the steps of: (1) preparing a liquid phase of a biologically active substance in a solvent or a mixture of solvents to which may be added one or more surfactants; (2) preparing a second liquid phase of a non-solvent or a mixture of non-solvents, the non-solvent is miscible with the solvent or mixture of solvents for the substance; (3) adding together the solutions of (1) and (2) with stirring; and (4) removing of unwanted solvents to produce a colloidal suspension of nanoparticles. The '528 patent describes that it produces particles of the substance smaller than 500 nm without the supply of energy.

Phase Inversion Precipitation

Another precipitation technique is phase inversion precipitation. One suitable phase inversion precipitation is described in U.S. Pat. Nos. 6,235,224, 6,143,211 and U.S. published patent application No. 2001/0042932. Phase inversion is a term used to describe the physical phenomena by which a polymer dissolved in a continuous phase solvent system inverts into a solid macromolecular network in which the polymer is the continuous phase. One method to induce phase inversion is by the addition of a nonsolvent to the continuous phase. The polymer undergoes a transition from a single phase to an unstable two phase mixture: polymer rich and polymer poor fractions. Micellar droplets of nonsolvent in the polymer rich phase serve as nucleation sites and become coated with polymer. The '224 patent describes that phase inversion of polymer solutions under certain conditions can bring about spontaneous formation of discrete microparticles, including nanoparticles. The '224 patent describes dissolving or dispersing a polymer in a solvent. A pharmaceutical agent is also dissolved or dispersed in the solvent. For the crystal seeding step to be effective in this process it is desirable the agent is dissolved in the solvent. The polymer, the agent and the solvent together form a mixture having a continuous phase, wherein the solvent is the continuous phase. The mixture is then introduced into at least tenfold excess of a miscible nonsolvent to cause the spontaneous formation of the microencapsulated microparticles of the agent having an average particle size of between 10 nm and 10 μm. The particle size is influenced by the solvent:nonsolvent volume ratio, polymer concentration, the viscosity of the polymer-solvent solution, the molecular weight of the polymer, and the characteristics of the solvent-nonsolvent pair. The process eliminates the step of creating microdroplets, such as by forming an emulsion, of the solvent. The process also avoids agitation and/or shear forces.

pH Shift Precipitation

Another precipitation technique is pH shift precipitation. pH shift precipitation techniques typically include a step of dissolving a drug in a solution having a pH where the drug is soluble, followed by the step of changing the pH to a point where the drug is no longer soluble. The pH can be acidic or basic, depending on the particular pharmaceutical compound. The solution is then neutralized to form a presuspension of submicron sized particles of the pharmaceutically active compound. One suitable pH shifting precipitation process is described in U.S. Pat. No. 5,665,331. The process includes the step of dissolving the pharmaceutical agent together with a crystal growth modifier (CGM) in an alkaline solution and then neutralizing the solution with an acid in the presence of suitable surface-modifying surface-active agent or agents to form a fine particle dispersion of the pharmaceutical agent. The precipitation step can be followed by steps of diafiltration clean-up of the dispersion and then adjusting the concentration of the dispersion to a desired level. This process reportedly leads to microcrystalline particles of Z-average diameters smaller than 400 nm as measured by photon correlation spectroscopy. Other examples of pH shifting precipitation methods are described in U.S. Pat. Nos. 5,716,642; 5,662,883; 5,560,932; and 4,608,278.

Infusion Precipitation Method

Another precipitation technique is infusion precipitation. Suitable infusion precipitation techniques are described in the U.S. Pat. Nos. 4,997,454 and 4,826,689. First, a suitable solid compound is dissolved in a suitable organic solvent to form a solvent mixture. Then, a precipitating nonsolvent miscible with the organic solvent is infused into the solvent mixture at a temperature between about −10° C. and about 100° C. and at an infusion rate of from about 0.01 ml per minute to about 1000 ml per minute per volume of 50 ml to produce a suspension of precipitated non-aggregated solid particles of the compound with a substantially uniform mean diameter of not greater than 10 μm. Agitation (e.g., by stirring) of the solution being infused with the precipitating nonsolvent is preferred. The nonsolvent may contain a surfactant to stabilize the particles against aggregation. The particles are then separated from the solvent. Depending on the solid compound and the desired particle size, the parameters of temperature, ratio of nonsolvent to solvent, infusion rate, stir rate, and volume can be varied according to the invention. The particle size is proportional to the ratio of nonsolvent: solvent volumes and the temperature of infusion and is inversely proportional to the infusion rate and the stirring rate. The precipitating nonsolvent may be aqueous or non-aqueous, depending upon the relative solubility of the compound and the desired suspending vehicle.

Temperature Shift Precipitation

Another precipitation technique is temperature shift precipitation. Temperature shift precipitation technique, also known as the hot-melt technique, is described in U.S. Pat. No. 5,188,837 to Domb. In an embodiment of the invention, lipospheres are prepared by the steps of: (1) melting or dissolving a substance such as a drug to be delivered in a molten vehicle to form a liquid of the substance to be delivered; (2) adding a phospholipid along with an aqueous medium to the melted substance or vehicle at a temperature higher than the melting temperature of the substance or vehicle; (3) mixing the suspension at a temperature above the melting temperature of the vehicle until a homogenous fine preparation is obtained; and then (4) rapidly cooling the preparation to room temperature or below.

Solvent Evaporation Precipitation

Another precipitation technique is solvent evaporation precipitation. Solvent evaporation precipitation techniques are described in U.S. Pat. No. 4,973,465. The '465 patent describes methods for preparing microcrystals including the steps of: (1) providing a solution of a pharmaceutical component and a phospholipid dissolved in a common organic solvent or combination of solvents, (2) evaporating the solvent or solvents and (3) suspending the film obtained by evaporation of the solvent or solvents in an aqueous solution by vigorous stirring. The solvent can be removed by adding energy to the solution to evaporate a sufficient quantity of the solvent to cause precipitation of the compound. The solvent can also be removed by other well known techniques such as applying a vacuum to the solution or blowing nitrogen over the solution.

Reaction Precipitation

Another precipitation technique is reaction precipitation. Reaction precipitation includes the steps of dissolving the pharmaceutical compound in a suitable solvent to form a solution. The compound should be added in an amount at or below the saturation point of the compound in the solvent. The compound is modified by reacting with a chemical agent or by modification in response to adding energy such as heat or UV light or the like to such that the modified compound has a lower solubility in the solvent and precipitates from the solution.

Compressed Fluid Precipitation

Another precipitation technique is compressed fluid precipitation. A suitable technique for precipitating by compressed fluid is described in U.S. Pat. No. 6,576,264. The method includes the steps of dissolving a water-insoluble drug in a solvent to form a solution. The solution is then sprayed into a compressed fluid, which can be a gas, liquid or supercritical fluid. The addition of the compressed fluid to a solution of a solute in a solvent causes the solute to attain or approach supersaturated state and to precipitate out as fine particles. In this case, the compressed fluid acts as an anti-solvent which lowers the cohesive energy density of the solvent in which the drug is dissolved.

Alternatively, the drug can be dissolved in the compressed fluid which is then sprayed into an aqueous phase. The rapid expansion of the compressed fluid reduces the solvent power of the fluid, which in turn causes the solute to precipitate out as fine particles in the aqueous phase. In this case, the compressed fluid acts as a solvent.

II. Other Approaches for Particle Preparation

In addition to approaches such as nanosuspension preparation, the particles of the present disclosure can also be prepared by mechanical grinding of the active agent. Mechanical grinding includes such techniques as jet milling, pearl milling, ball milling, hammer milling, fluid energy milling or wet grinding techniques such as those described in U.S. Pat. No. 5,145,684.

Another method to prepare the particles is by suspending an active agent. In this method, particles of the active agent are dispersed in an aqueous medium by adding the particles directly into the aqueous medium to derive a pre-suspension. The particles are normally coated with a surface modifier to inhibit the aggregation of the particles. One or more other excipients can be added either to the active agent or to the aqueous medium.

III. Nanoparticles for Minimizing Drug-Drug Interaction

Generally, a pharmaceutical component in nanoparticle form will be sequestered by fixed macrophages within the RES or MPS, whereas a pharmaceutical component in solution form is absorbed and distributed systemically. More specifically, upon parenteral administration, a pharmaceutical component in nanoparticle form is generally not readily soluble in the blood, and is recognized as being a foreign body requiring elimination from systemic circulation. Accordingly, the pharmaceutical component in nanoparticle form is sequestered by fixed macrophages in the RES or MPS via phagocytosis. Enveloped in the fixed macrophages, the pharmaceutical component in nanoparticle form dissolves therefrom, enabling it to migrate out of the phagolysozomes and then to the extracellular milieu.

In this way, the phagocytosis and dissolution from the fixed macrophages causes the pharmaceutical component in nanoparticles form to have a metabolic timing which is different from the metabolic timing of the pharmaceutical component in solution form. Accordingly, the rate of dissolution and release by the macrophages within the RES or MPS and, in effect, the drug-drug interaction between pharmaceutical components may be controlled by administering a pharmaceutical component in the form of a nanoparticle (e.g., in nanosuspension form) with a pharmaceutical component in the form of a solution, in order to minimize drug-drug interaction between the components.

Generally, pharmaceutical components in nanoparticle form include molecules that are aggregated as a crystal or in an amorphous state. Such aggregation must be disassembled (“dissolved”) in the MPS before the molecules are capable of exiting to the extracellular milieu. In order to enhance the likelihood of phagocytosis ensuing, it is typically preferable that the nanoparticles in a nanosuspension have a crystalline form or characteristics. Specifically, nanoparticles associated with a crystalline lattice are more likely to resist solubilization and, therefore, systemic absorption and distribution, than are nanoparticles or other materials in an amorphous form. Nanoparticles in an amorphous form are typically less capable of resisting solubilization. As such, amorphous forms of nanoparticles are often absorbed and distributed systemically. However, in some cases, amorphous nanoparticles may be taken up by the RES or MPS. In some situations, amorphous forms of nanoparticles may be reformulated into crystalline form.

Microemulsions

The pharmaceutical component having a modulated pharmacokinetic profile may also be provided in the form of microemulsions. Microemulsions are modified vehicles of delivering pharmaceutical components comprised generally of water, oil and surfactant(s), which constitute a single optically isotropic and thermodynamically stable liquid solution. The size of microemulsion droplets ranges from about 10-100 nm. Microemulsions have the capacity to solubilize both water-soluble and oil-soluble compounds. Accordingly, for delivery, microemulsions can be comprised of oil droplets in an aqueous continuum, water in an oil continuum, or a bicontinuous structure, referred to as cubosomes.

The diffusion and release of hydrophobic drugs is slower than that of water-soluble drugs for oil-in-water microemulsions, whereas the opposite is true for water-in-oil microemulsions. Therefore, for minimizing drug-drug interaction, the absorption and distribution of microemulsions may be altered by adjusting the oil/water partitioning.

Because of the presence of oil, microemulsions are not readily soluble in the blood, and are recognized as being a foreign body requiring elimination from systemic circulation. Accordingly, microemulsions are sequestered by fixed macrophages in the RES or MPS via phagocytosis. Enveloped in the fixed macrophages, microemulsions dissolve therefrom, enabling the pharmaceutical component to migrate out of the phagolysozomes and then to the extracellular milieu.

Due to the sequestration by and egress from the MPS system, the pharmacokinetic profile of a pharmaceutical component in a microemulsion form is altered from the pharmacokinetic profile of the component in a non-microemulsion form. Accordingly, a drug-drug interaction may be reduced through modulating the pharmacokinetic profile of pharmaceutical component by formulating the component into a microemulsion.

In preparing an emulsion formulation having a modulated pharmacokinetic profile, one suitable emulsion precipitation technique is described in the co-pending and commonly assigned U.S. Patent Application Publication No. 2005/0037083. In this approach, the process includes the steps of: (1) providing a multiphase system having an organic phase and an aqueous phase, the organic phase having a pharmaceutically effective compound therein; and (2) sonicating the system to evaporate a portion of the organic phase to cause precipitation of the compound in the aqueous phase and having an average effective particle size of not greater than about 2 μm. The step of providing a multiphase system includes the steps of: (1) mixing a water immiscible solvent with the pharmaceutically effective compound to define an organic solution, (2) preparing an aqueous based solution with one or more surface active compounds, and (3) mixing the organic solution with the aqueous solution to form the multiphase system. The step of mixing the organic phase and the aqueous phase can include the use of piston gap homogenizers, colloidal mills, high speed stirring equipment, extrusion equipment, manual agitation or shaking equipment, microfluidizer, or other equipment or techniques for providing high shear conditions. The crude emulsion will have oil droplets in the water of a size of approximately not greater than 1 μm in diameter. The crude emulsion is sonicated to define a microemulsion and eventually to define a submicron sized particle suspension.

Another approach to preparing an emulsion having submicron-sized particles is disclosed in co-pending and commonly assigned U.S. Patent Application Publication No. 2003/0059472. The process includes the steps of: (1) providing a crude dispersion of a multiphase system having an organic phase and an aqueous phase, the organic phase having a pharmaceutical compound therein; (2) providing energy to the crude dispersion to form a fine dispersion; (3) freezing the fine dispersion; and (4) lyophilizing the fine dispersion to obtain submicron sized particles of the pharmaceutical compound. The step of providing a multiphase system includes the steps of: (1) mixing a water immiscible solvent with the pharmaceutically effective compound to define an organic solution; (2) preparing an aqueous based solution with one or more surface active compounds; and (3) mixing the organic solution with the aqueous solution to form the multiphase system. The step of mixing the organic phase and the aqueous phase includes the use of piston gap homogenizers, colloidal mills, high speed stirring equipment, extrusion equipment, manual agitation or shaking equipment, microfluidizer, or other equipment or techniques for providing high shear conditions.

Generally, a pharmaceutical component in microemulsion form has a faster rate of dissolution within the RES or MPS than a pharmaceutical component in nanoparticle form. The faster rate is because a pharmaceutical component in microemulsion form is phagocytized by the MPS but the molecules of the pharmaceutical component in the microemulsion are not in an aggregated, and hence less-soluble form. In contrast, the pharmaceutical component in nanoparticle form contains molecules that are aggregated as a crystal or in an amorphous state, and such aggregation must be disassembled (“dissolved”) in the MPS before exiting to the extracellular milieu. In further contrast, a pharmaceutical component in a conventional solution form is rapidly distributed systemically. Accordingly, the rate of dissolution and release by the macrophages within the RES or MPS and, in effect, the drug-drug interaction between pharmaceutical components may be controlled using varying the vehicles of delivery. For example, a pharmaceutical component in the form of a microemulsion may be administered with another pharmaceutical component in the form of nanoparticles to provide a pharmaceutical combination having reduced drug-drug interaction. Alternatively, a pharmaceutical microemulsion may be administered with another pharmaceutical component in the form of a solution, in order to minimize drug-drug interaction between the components.

Emulsions

The pharmaceutical component having a modulated pharmacokinetic profile may also be provided in the form of emulsions. Emulsions comprise droplets which are relatively large in size as compared to microemulsions. In contrast to microemulsions which form spontaneously, emulsions must be prepared with the input of energy. Formation of emulsions includes high pressure homogenization for producing emulsion droplets (ranging in size from about 100 nm-10 μm) and generating a new surface thereon. Emulsions may be water-in-oil or oil-in-water based on surfactants, oil and water volume fraction, temperature, salt concentration, and the presence of cosurfactants and other cosolutes. Multiple emulsions comprising a water-in-oil-in-water or oil-in-water-in-oil may further be formed via a double homogenization process.

Due to the relatively large size of the oil droplets, an oil-in-water type emulsion has a relatively large hydrophobic volume in comparison to the oil-in-water surface area. This relationship allows for large amounts of hydrophobic active ingredients to be incorporated in oil-in-water emulsions. Moreover, because, the surface area is not large, the amount of surfactant required for generating and stabilizing emulsions is comparatively low, and nontoxic surfactants, such as phospholipids and other polar lipids, can be used as stabilizers.

The emulsion droplets may be formulated so as not to be readily soluble in the blood, and allow time to be recognized as being a foreign body requiring elimination from systemic circulation. For example, emulsions typically degrade within an hour after injection. Longer lived emulsions that can be phagocytized could be prepared however. Accordingly, this modified formulation of emulsions is sequestered by fixed macrophages in the RES or MPS via phagocytosis. Enveloped in the fixed macrophages, emulsions dissolve therefrom, enabling the drug molecules to migrate out of the phagolysozomes and then to the extracellular milieu.

In this way, the phagocytosis and dissolution from the fixed macrophages causes emulsions to have a metabolic timing which is different from the metabolic timing of the pharmaceutical component in solution form. In yet another embodiment, drug-drug interaction may be reduced through modulating the pharmacokinetic profile of pharmaceutical components by incorporating them into emulsions by manipulating the component of the emulsion and the surface modifiers thereon.

Generally, a pharmaceutical component in emulsion form has a faster rate of dissolution within the RES or MPS than a pharmaceutical component in nanoparticle form. The faster rate is because a pharmaceutical component in emulsion form is phagocytized by the MPS but the molecules of the pharmaceutical component in the emulsion are not in an aggegrated form. In contrast, the pharmaceutical component in the nanoparticle form contains molecules that are aggregated as a crystal or in an amorphous state, and such aggregation must be disassembled before the molecules exit to the extracellular milieu. In further contrast, a pharmaceutical component in solution form is absorbed and distributed systemically. Accordingly, the rate of dissolution and release by the macrophages within the RES or MPS and, in effect, the drug-drug interaction between pharmaceutical components may be controlled by varying the vehicles of delivery. For example, a pharmaceutical component in the form of an emulsion may be administered with another pharmaceutical component in the form of nanoparticles to provide a pharmaceutical combination having reduced drug-drug interaction. Alternatively, a pharmaceutical emulsion may be administered with another pharmaceutical component in the form of a solution, in order to minimize drug-drug interaction between the components.

Micelles

The pharmaceutical component having a modulated pharmacokinetic profile may also be provided in the form of micelles. Micelles are modified vehicles of delivering pharmaceutical components comprising a conglomeration of surfactant molecules. Formation of micelles is generally dictated by the interaction between the hydrophobic parts of the surfactant molecules. Interactions opposing micellization include electrostatic repulsive interactions between charged head groups of ionic surfactants, repulsive osmotic interactions between chainlike polar head groups such as oligo chains, or steric interactions between bulky head groups. In maintaining the balance between the opposing forces, micelle formation is dependent on size of the hydrophobic moiety, the nature of the polar head group, the nature of the counterion (charged surfactants, salt concentration), pH, temperature, and presence of cosolutes. For example, an increase in size of the hydrophobic domain causes increased hydrophobic interaction, thereby causing micellization.

Micelles form highly dynamic structures whereupon the molecules therein remain in a generally non-aggregated state. Furthermore, in solution, surfactant molecules freely exchange among individual micelles. Solubility of a hydrophobic drug is dependent on the number and aggregation of micelles. Accordingly, larger micelles are generally more efficient solubilizers of hydrophobic drugs than smaller micelles. Micelles comprising low molecular weight surfactants may disassociate rapidly after parenteral administration. On the other hand, micelles comprising high molecular weight surfactants, a higher concentration of surfactant, and micelles formed in block copolymer form may delay disassociation, permitting time for them to be recognized as foreign and thus be phagocytized.

Thus micelles may be formulated so as to be not readily soluble in the blood and are recognized as being a foreign body requiring elimination from systemic circulation. Accordingly, micelles are sequestered by fixed macrophages in the RES or MPS via phagocytosis. Enveloped in the fixed macrophages, micelles dissolve therefrom, enabling the pharmaceutical component to migrate out of the phagolysozomes and then to the extracellular milieu. In this way, the phagocytosis and dissolution from the fixed macrophages causes micelles to have a metabolic timing which is different from the metabolic timing of the pharmaceutical component in solution form. Accordingly, drug-drug interaction may be reduced through modulating the pharmacokinetic profile of micelles by manipulating the structure of the micellular structures.

Liposomes

The pharmaceutical component having a modulated pharmacokinetic profile may also be provided in the form of liposomes. Liposomes are modified vehicles of delivering pharmaceutical components comprising a conglomeration of surfactant molecules and sometimes block polymers having one or several bilayer structures, normally comprising lipid. Liposomes possess the capability to incorporate both water-soluble and oil-soluble substances.

Drug release in liposomes generally involves the manipulating the permeability of the lipid bilayers by (1) altering the component (s) of the lipid bilayers, (2) altering pH, (3) removing bilayer components, and (4) introducing a complement component. Nevertheless, liposomes are not readily absorbed and distributed while residing in systemic circulation after initial administration.

More specifically, liposomes are not readily soluble in the blood, and are recognized as being a foreign bodies requiring elimination from systemic circulation. Accordingly, liposomes are sequestered by fixed macrophages in the RES or MPS via phagocytosis. Enveloped in the fixed macrophages, liposomes dissolve therefrom, enabling the pharmaceutical component to migrate out of the phagolysozomes and then to the extracellular milieu.

In this way, the phagocytosis and dissolution from the fixed macrophages causes liposomes to have a metabolic timing which is different from the metabolic timing of the pharmaceutical component in solution form. Accordingly, drug-drug interaction may be reduced through modulating the pharmacokinetic profile of liposomes by manipulating their component.

Generally, a pharmaceutical component in the form of liposomes has a faster rate of dissolution within the RES or MPS than a pharmaceutical component in nanoparticle form which is subject to phagocytosis. The faster rate is because the pharmaceutical component is incorporated into the liposomes in the molecularly dissolved state whereas the pharmaceutical component in nanoparticle form contains molecules in an aggregated form and requires an initial dissolution step in the MPS. In further contrast, a pharmaceutical component in solution form avoids phagocytosis and is distributed systemically. Accordingly, the pharmacokinetic profile and, in effect, the drug-drug interaction between pharmaceutical components may be controlled using varying the vehicles of delivery. For example, a pharmaceutical component in the form of a liposome may be administered with a pharmaceutical component in the form of a nanoparticle, alternatively in the form of a sized micelle, or alternatively in the form of a solution, in order to minimize drug-drug interaction between the components.

Combining the Use of Multiple Modified Drug Delivery Vehicles

Pharmaceutical components having different modified drug delivery vehicles may be used for achieving minimization of drug-drug interaction between such components. In one aspect of the invention, multiple drug delivery vehicles may be used to minimize drug-drug interaction between a plurality of pharmaceutical components. In this case, a first pharmaceutical component is provided having a particular pharmacokinetic profile based in part by its drug delivery state. For example, the first pharmaceutical component may be delivered in the form of a nanoparticle, nanosuspension, microemulsion, emulsion, micelle, or liposome. The first pharmaceutical component may further be delivered in solution form when the second pharmaceutical component is not in solution form. A second pharmaceutical component is further provided having another pharmaceutical profile based in part by its drug delivery state. The second pharmaceutical component may be delivered in the form of a nanoparticle, nanosuspension, microemulsion, emulsion, micelle, or liposome. The second pharmaceutical component may further be delivered in solution form when the first pharmaceutical component is not in solution form. The drug delivery vehicles are selected such that the first and second pharmaceutical components do not substantially affect each other or at least the interaction between their respective profiles is substantially reduced compared to unmodified formulation states of the component of the combination that is in the modified delivery state.

For example, generally, nanosuspensions, microemulsions, emulsions, micelles, and liposomes have varying rates of dissolution and release by the macrophages within the RES or MPS. In one more specific example, the rate of dissolution of the liposomes is generally faster than nanosuspensions, which provides a longer release time for the pharmaceutical component in nanosuspension form. Therefore, a pharmaceutical combination may be provided including at least one pharmaceutical component formulated in nanosuspension form having a certain modulated pharmacokinetic profile of concentration variation over time. A second pharmaceutical component formulated in liposomal form having a different modulated pharmacokinetic profile of concentration variation over time may further be provided. When administering this pharmaceutical combination to a mammal either at about the same time or at staggered times or in the same or separate delivery compositions, the rate of dissolution of liposomes in the MPS/RES is faster than the rate of dissolution of nanosuspensions. Accordingly, one or more of the pharmaceutical components is formulated to have an altered pharmacokinetic profile, and these components are administered in this manner so as to reduce drug-drug interactions that would occur when administering compositions having only unmodified formulated states.

### EXAMPLE 1

FIG. 3 illustrates a modulated pharmacokinetic profile resulting in minimization of drug-drug interaction with an itraconazole nanosuspension. This plots the release of a nanosuspension itraconazole, designated at 10, as compared to liquid injectable itraconazole, designated at 12. The itraconazole formulation illustrated in FIG. 3 is the Sporanox® brand intravenous injection solution manufactured by Janssen Pharmaceutica Products, L.P. For each of the nanosuspension itraconazole component 10 and the liquid injectable Sporanox® itraconazole component 12, 10 mg/mL are administered. The initial decline of the plot supports an observation that the liquid injectable Sporanox® itraconazole component 12 is rapidly removed from systemic circulation. Further data along the plot supports an observation that the nanosuspension itraconazole component 10 is also rapidly removed from systemic circulation due to phagocytosis by the RES or MPS.

Plot 10 of FIG. 3 also is consistent with observations that the nanosuspension itraconazole component 10 is sequestered and enveloped by the fixed macrophages of the RES or MPS as depicted by a decrease of nanosuspension concentration. The reported increase in nanosuspension concentration thereafter supports a conclusion that the nanosuspension itraconazole then dissolves therefrom, enabling it to migrate out of the phagolysozomes and then to the extracellular milieu. A second slower decline in nanosuspension concentration is consistent with a gradual metabolism of the nanosuspension. Overall, the data of FIG. 3 supports the conclusion that nanosuspension phagocytosis takes place. See “Long-Circulating and Target-Specific Nanoparticles: Theory to Practice,” S. Moein Moghimi et al., Pharmacological Reviews, Vol. 53, No. 2 (2001) and “Nanosuspensions in Drug Delivery,” Barrett E. Rabinow, Nature, Vol. 3, (September 2004).

The itraconazole in a nanosuspension formulation effectively causes the pharmacokinetic profile of plasma concentration variation over time to be modified compared to Sporanox® itraconazole. For example, there is a decrease in peak plasma concentration level (Cₘₐₓ) for the nanosuspension formulation as compared to the solution form. Also, the peak plasma concentration level (Cₘₐₓ) occurs at different points over the same time period for both formulations. More specifically, the Cₘₐₓ in the plasma curve of the nanosuspension occurs not immediately after injection as it does with liquid injectable forms, but several hours later following the phagocytosis and release of the nanoparticles from the macrophages of the RES or MPS.

Therefore, a pharmaceutical combination may be provided including itraconazole in nanosuspension form having a certain modulated rate of release and altered pharmacokinetic profile. The pharmaceutical combination of may further include another pharmaceutical component in liquid injectable form. In this way, the potential drug-drug interaction between the itraconazole formulation and the other pharmaceutical component is minimized by providing the itraconazole formulation in nanosuspension form thereby altering the rate of dissolution or release of this itraconazole formulation by the RES or MPS.

Equation 1 illustrates a mathematical representation of drug metabolic inhibition factors (R).
R=1+fᵤ*C_{max,I,L}/Kᵢ Equation 1

Drug metabolic inhibition factors (R) indicate the factor by which the concentration of drug may be increased by a co-administered drug that interferes with the metabolism of the first drug.

In Equation 1, fᵤ represents the fraction of inhibitor drug unbound in plasma, wherein the unbound drug is free to equilibrate out of the blood compartment across membranes into the tissues. Kᵢ represents the inhibition constant of the inhibitor for the drug whose concentration is being affected. C_{max,I,L} represents the liver Cₘₐₓ of the inhibitor after administration. C_{max,I,L} typically calculated by multiplying Cₘₐₓ of the inhibitor in plasma determined in a pharmacokinetic study (C_{max,I,P}), by the liver/plasma concentration ratio, determined in a tissue distribution study.

An example for comparison of drug inhibition factors calculated for Sporanox® itraconazole component as a solution versus an itraconazole nanosuspension formulation is as follows using midazolam as the second, affected drug

For humans, the Sporanox® itraconazole component as a solution has a C_{max,I,P}=3748 ng/ml. The liver/plasma concentration ratio (P_{L}) is 3.5. Therefore, C_{max,I,L}=13118 ng/ml. For a 200 mg dose of itraconazole, fᵤ=0.035. For midazolam, the Kᵢ=0.275 μM.

For dogs, the Sporanox® itraconazole component as a solution has a C_{max,I,P}=3 μg/ml. For the nanosuspension formulation of the itraconazole component, the Cₘₐₓ in the plasma curve occurs not immediately after injection as it does with solution dosage forms, but several hours later following the phagocytosis and release from the macrophages of the liver, as discussed in detail with regard to FIG. 3. Accordingly, the C_{max,I,P}=0.31 μg/ml for the nanosuspension formulation, which includes the hydroxy-itraconazole metabolite in addition to the itraconazole. With these values in mind, Sporanox® itraconazole has a plasma C_{max,I,L} of 10.5 μg/ml, whereas the nanosuspension formulation of itraconazole has a C_{max,I,P} of 1.085 μg/ml for both parent and metabolite. Accordingly, the drug metabolic inhibition factor for Sporanox® is (R)=1+0.035(10.5/0.275)=2.35. The inhibition constant R for the nanosuspension formulation of itraconazole upon midazolam is given as: R=1+0.035(1.085/0.275)=1.14. From this mathematical representation, Sporanox® will increase the concentration of midazolam by a significant factor (2.35) compared with the negligible increase caused by the nanosuspension formulation of itraconazole (1.14). Thus, the concentration of itraconazole in a nanosuspension form may be increased to increase efficacy, without concern for increasing drug-drug interaction.

### EXAMPLE 2

This Example illustrates reducing drug-drug interaction with itraconazole in a modified drug delivery formulation. When concomitantly administered with various other drugs and not according to the invention, Sporanox® itraconazole increases plasma concentrations of certain drugs. These drugs include antiarrhythmics (e.g., digoxin, dofetilide, quinidine, disopyramide), anticonvulsants (e.g, carbamazepine), antimycobacterials (e.g., rifabutin), antineoplastics (e.g., busulfan, docetaxel, vinca alkaloids), antipsychotics (e.g., pimozide), benzodidiazepines (e.g., alprazolam, diazepam, midazolam, or triazolam), calcium channel blockers (e.g., dihydropyridines, verapamil), gastrointestinal motility agents (e.g., cisapride), HMG co-a-reductase inhibitors (e.g., atorvastatin, cerivastatin, lovastatin, simvastatin), immunosuppressants (e.g., cyclosporine, tacrolimus, sirolimus), oral hypoglycemics, protease inhibitors (idinavir, ritonavir, saquinavir), levacetylmethadol (levomethadyl), ergot alkaloids, halofantrins, alfentanil, buspirone, methylprednisolone, budesonide, dexamethsone, trimetrexate, warfarin, cilostazol, and cletripan. The side-effects associated with this drug-drug interaction include, among other reactions, serious cardiovascular events, prolonged hypnotic and sedative effects, and cerebral ischemia. Therefore, in accordance with the teachings of this invention, the formulation of Sporanox® itraconazole is modified in order to minimize drug-drug interaction with the above-listed drugs.

More specifically, the pharmacokinetic profile and, in effect, the drug-drug interaction between Sporanox® itraconazole and each of the above-listed drugs is reduced by using varying modified vehicles for delivering itraconazole. In this Example, itraconazole in the form of a nanosuspension is concomitantly administered with digoxin in order to reduce drug-drug interaction. Other concomitant administrations are with itraconazole nanosuspension and each of the other drugs listed above.

Alternatively, itraconazole in the form of nanoparticles, nanosuspensions, emulsions, micelles, and liposomes each have varying rates of dissolution or release within the RES or MPS. Accordingly, itraconazole is administered in the form of any one of an emulsion, microemulsion liposome, or micelle, concomitantly administered with the above-listed drugs in order to reduce drug-drug interaction (e.g., digoxin+microemulsion, emulsion, liposome, or micelle forms of itraconazole).

### EXAMPLE 3

This Example concerns reduction of drug-drug interaction between Sporanox® itraconazole and a pharmaceutical component in a modified drug delivery formulation. When concomitantly administered, not according to the invention, certain drugs increase plasma concentrations of itraconzaole. These drugs include macrolide antibiotics (e.g., clarithromycin, erythromycin) and protease inhibitors (indinavir, ritonavir). In accordance with the teachings of this disclosure, the formulation of these drugs is modified in order to reduce drug-drug interaction with Sporanox® itraconazole. More specifically, the pharmacokinetic profile is altered by using varying vehicles for delivering the above-listed drugs. In effect, the drug-drug interaction between Sporanox® itraconazole and each of the above-listed drugs in a modified delivery form is reduced.

A nanosuspension of clarithromycin is concomitantly administered with Sporanox® itraconazole (in solution form) to reduce drug-drug interaction therebetween when compared with clarithromycin in an unmodified delivery form. Alternatively, an above-listed drug in the form of emulsions, micelles, or liposomes is concomitantly administered with Sporanox® itraconazole in order to reduce drug-drug interaction.

While this invention has been described with reference to certain illustrative aspects, it will be understood that this description shall not be construed in a limiting sense. Rather, various changes and modifications can be made to the illustrative embodiments, including various combinations of specific aspects thereof, without departing from the true spirit, central characteristics and scope of the invention, including those combinations of features that are individually disclosed or claimed herein. Furthermore, it will be appreciated that any such changes and modifications will be recognized by those skilled in the art as an equivalent to one or more elements of the following claims, and shall be covered by such claims to the fullest extent permitted by law.

1. A pharmaceutical combination for minimizing pharmacokinetic drug-drug interaction within a mammal, the pharmaceutical combination comprising:
a first pharmaceutical component having a particular pharmacokinetic profile in the mammal; and
a second pharmaceutical component formulated for parenteral administration, said second pharmaceutical component being formulated such that the pharmacokinetic profile of said second pharmaceutical component is altered from its unaltered pharmacokinetic profile, which unaltered profile substantially affects said particular pharmacokinetic profile of the first pharmaceutical component, so that said altered pharmacokinetic profile of said second pharmaceutical component does not substantially affect the pharmacokinetic profile of said first pharmaceutical component.

2. The pharmaceutical combination for minimizing pharmacokinetic drug-drug interaction of claim 1, wherein the second pharmaceutical component is insoluble.

3. The pharmaceutical combination for minimizing pharmacokinetic drug-drug interaction of claim 2, wherein the second pharmaceutical component is administered with a drug delivery vehicle modification.

4. The pharmaceutical combination for minimizing pharmacokinetic drug-drug interaction of claim 3, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

5. The pharmaceutical combination for minimizing pharmacokinetic drug-drug interaction of claim 1, wherein the second pharmaceutical component is phagocytized in the MPS of the mammal.

6. The pharmaceutical combination for minimizing pharmacokinetic drug-drug interaction of claim 1, wherein the second pharmaceutical component is administered with a micelle drug delivery vehicle modification, wherein the pharmacokinetic profile of the second pharmaceutical component is altered by its association with the micelle.

7. The pharmaceutical combination for minimizing pharmacokinetic drug-drug interaction of claim 1, wherein the second pharmaceutical component is administered with a microemulsion drug delivery vehicle modification, said microemulsion comprising an oil/water partition wherein the pharmacokinetic profile of the second pharmaceutical component is altered by its formulation as a microemulsion with the oil/water partition.

8. The pharmaceutical combination for minimizing pharmacokinetic drug-drug interaction of claim 1, wherein the second pharmaceutical component is administered with an emulsion drug delivery vehicle modification, said emulsion comprising an oil/water partition wherein the pharmacokinetic profile of the second pharmaceutical component is altered by its formulation as an emulsion.

9. The pharmaceutical combination for minimizing pharmacokinetic drug-drug interaction of claim 3, wherein the drug delivery vehicle modification further comprises surface modifiers and the pharmacokinetic profile of the second pharmaceutical component is altered by its association with surface modifiers.

10. The pharmaceutical combination for minimizing pharmacokinetic drug-drug interaction of claim 3, wherein the drug delivery vehicle modification is a nanosuspension of crystalline nanoparticles.

11. The pharmaceutical combination for minimizing pharmacokinetic drug-drug interaction of claim 3, wherein the drug delivery vehicle modification is a nanosuspension of amorphous nanoparticles.

12. The pharmaceutical combination for minimizing pharmacokinetic drug-drug interaction of claim 10, wherein the second pharmaceutical component is itraconazole.

13. The pharmaceutical combination for minimizing pharmacokinetic drug-drug interaction of claim 1, wherein the pharmacokinetic profiles of the first and second pharmaceutical components are measured by plasma concentration variation over time; and the modified formulated second pharmaceutical component, when administered to the mammal, has a pharmacokinetic profile of plasma concentration variation over time different from the pharmacokinetic profile of the second pharmaceutical component in an unmodified formulated state over the same time period, wherein the different plasma concentration variation minimizes pharmacokinetic drug-drug interaction between the first and second pharmaceutical components when said first and second pharmaceutical components concurrently reside within the mammal.

14. The pharmaceutical combination for minimizing pharmacokinetic drug-drug interaction of claim 13, wherein the unaltered second pharmaceutical component has a peak plasma concentration at a certain point over a period of time and the altered second pharmaceutical component has a peak plasma concentration occurring at a different point over the same period of time due to its modified formulation.

15. The pharmaceutical combination for minimizing pharmacokinetic drug-drug interaction of claim 13, wherein the unaltered second pharmaceutical component has a peak plasma concentration, and the altered second pharmaceutical component has a peak plasma concentration which is lower than the peak plasma concentration of the unaltered second pharmaceutical component.

16. The pharmaceutical combination for minimizing pharmacokinetic drug-drug interaction of claim 13, wherein the pharmacokinetic profile of concentration variation over time of said second pharmaceutical component is associated with the phagocytosis of the second pharmaceutical component by macrophages in the MPS after administration to the mammal.

17. The pharmaceutical combination for minimizing pharmacokinetic drug-drug interaction of claim 13, wherein the first pharmaceutical component has a plasma concentration at any given point in time and the second pharmaceutical component in the modified formulation has a lower plasma concentration, than it would have in an unmodified formulated state, so as to reduce the total concentration of pharmaceutical components at said given point in time.

18. The pharmaceutical combination for minimizing pharmacokinetic drug-drug interaction of claim 13, wherein the given formulation of the second pharmaceutical component exhibits a given average plasma concentration over a certain period of time when administered at a selected dose, and wherein the modified second pharmaceutical component exhibits a lower average plasma concentration over a longer period of time when administered at the same selected dose.

19. A method for minimizing drug-drug interaction in a mammal comprising:
administering a first pharmaceutical component having a particular pharmacokinetic profile in the mammal;
providing a second pharmaceutical component, the second component in a given formulation having a particular pharmacokinetic profile in the mammal, wherein the particular pharmacokinetic profile of the second pharmaceutical component in the given formulation substantially affects the pharmacokinetic profile of the first pharmaceutical component when the first and second pharmaceutical components concurrently reside within the mammal;
formulating the second pharmaceutical component into a modified formulation, wherein the modified formulation changes the particular pharmacokinetic profile of the second pharmaceutical component into an altered pharmacokinetic profile; and
administering the modified formulation of the second pharmaceutical component to the mammal parenterally, wherein the altered pharmacokinetic profile of the second component has a substantially reduced effect, compared to the effect of the second pharmaceutical component given formulation, on the pharmacokinetic profile of the first pharmaceutical component when the first pharmaceutical component and the second pharmaceutical component concurrently reside within the mammal.

20. The method for minimizing drug-drug interaction in a mammal of claim 19, wherein the altered pharmacokinetic profile of the second component does not substantially affect the pharmacokinetic profile of the first pharmaceutical component.

21. The method for minimizing drug-drug interaction in a mammal of claim 19, wherein the second pharmaceutical component is insoluble.

22. The method for minimizing drug-drug interaction in a mammal of claim 20, wherein the formulation of the second pharmaceutical component is modified via a drug delivery vehicle modification.

23. The method for minimizing drug-drug interaction in a mammal of claim 22, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

24. The method for minimizing drug-drug interaction in a mammal of claim 19, wherein the first pharmaceutical component, when administered to the mammal, has a particular pharmacokinetic profile as measured by plasma concentration variation over time; and the second pharmaceutical component in the modified formulation, when administered to the mammal, has a pharmacokinetic profile of as measured by plasma concentration variation over time different from that of the second pharmaceutical component in the unmodified formulation over the same time period, wherein the different plasma concentration variation minimizes pharmacokinetic drug-drug interaction between the first and second pharmaceutical components when said first and second pharmaceutical components concurrently reside within the mammal.

25. The method for minimizing drug-drug interaction in a mammal of claim 24, wherein the first pharmaceutical component has a plasma concentration at any given point in time and the second pharmaceutical component in the modified formulation has a lower plasma concentration, than it would have in an unmodified formulated state, so as to reduce the total concentration of pharmaceutical components at said given point in time.

26. The method for minimizing drug-drug interaction in a mammal of claim 25, wherein the given formulation of the second pharmaceutical component exhibits a given average plasma concentration over a certain period of time when administered at a selected dose, and wherein the modified second pharmaceutical component exhibits a lower average plasma concentration over a longer period of time when administered at the same selected dose.

27. The method for minimizing drug-drug interaction in a mammal of claim 25, wherein the second pharmaceutical component in the unmodified formulation has a peak plasma concentration, and the second pharmaceutical component in the modified formulation has a peak plasma concentration which is lower than the peak plasma concentration of the second pharmaceutical component in the unmodified formulation.

28. The method for minimizing drug-drug interaction in a mammal of claim 25, wherein the pharmacokinetic profile of concentration variation over time of said second pharmaceutical component in the modified formulation is associated with the phagocytosis of the second pharmaceutical component in the modified formulation by macrophages in the MPS after administration to the mammal.

29. A method for minimizing drug-drug interaction in a mammal comprising:
providing a first pharmaceutical component having a particular pharmacokinetic profile in the mammal;
providing a second pharmaceutical component, the second component in a given formulation having a particular pharmacokinetic profile in the mammal, wherein the particular pharmacokinetic profile of the second pharmaceutical component substantially affects the pharmacokinetic profile of the first pharmaceutical component when the first and second pharmaceutical components concurrently reside within the mammal;
formulating the second pharmaceutical component into a modified formulation, wherein the modified formulation changes the particular pharmacokinetic profile of the second pharmaceutical component into an altered pharmacokinetic profile;
administering the modified second pharmaceutical component to the mammal parenterally; and
administering the first pharmaceutical component to the mammal, wherein the pharmacokinetic profile of the modified formulation of the second pharmaceutical component substantially minimizes the effect on the pharmacokinetic profile of the first pharmaceutical component when the first pharmaceutical component and the second pharmaceutical component concurrently reside within the mammal.

30. The method for minimizing drug-drug interaction in a mammal of claim 29, wherein the altered pharmacokinetic profile of the second component does not substantially affect the pharmacokinetic profile of the first pharmaceutical component.

31. The method for minimizing drug-drug interaction in a mammal of claim 30, wherein the second pharmaceutical component is insoluble.

32. The method for minimizing drug-drug interaction in a mammal of claim 31, wherein the formulation of the second pharmaceutical component is modified via a drug delivery vehicle modification.

33. The method for minimizing drug-drug interaction in a mammal of claim 32, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

34. The method for minimizing drug-drug interaction in a mammal of claim 30, wherein the second pharmaceutical component in the unmodified formulation, when administered to the mammal, has a particular pharmacokinetic profile as measured byplasma concentration variation over time; and the second pharmaceutical component in the modified formulation, when administered to the mammal, has a pharmacokinetic profile as measured by plasma concentration variation over time different from the second pharmaceutical component in the unmodified formulation over the same time period, wherein the different plasma concentration variation minimizes pharmacokinetic drug-drug interaction between the first and second pharmaceutical components when said first and second pharmaceutical components concurrently reside within the mammal.

35. The method for minimizing drug-drug interaction in a mammal of claim 34, wherein the second pharmaceutical component in the unmodified formulation has a peak plasma concentration at a certain point over a period of time and the second pharmaceutical component in the modified formulation has a peak plasma concentration occurring at a different point over the same period of time.

36. The method for minimizing drug-drug interaction in a mammal of claim 35, wherein the second pharmaceutical component in the unmodified formulation has a peak plasma concentration, and the second pharmaceutical component in the modified formulation has a peak plasma concentration which is lower than the peak plasma concentration of the second pharmaceutical component in the unmodified formulation.

37. The method for minimizing drug-drug interaction in a mammal of claim 34, wherein the pharmacokinetic profile of concentration variation over time of said second pharmaceutical component in the modified formulation is associated with the phagocytosis of the second pharmaceutical component in the modified formulation by macrophages in the MPS after administration to the mammal.

38. A pharmaceutical combination for minimizing pharmacokinetic drug-drug interaction within a mammal, the pharmaceutical combination comprising:
a first pharmaceutical component that is metabolized by a particular drug-metabolizing mechanism according to a specific metabolic timing, and
a second pharmaceutical component that is phagocytized in the MPS, said second pharmaceutical component being metabolized by a similar drug-metabolizing mechanism as the first pharmaceutical component, wherein phagocytosis of the second pharmaceutical component results in a metabolic timing which is different from the metabolic timing of the first pharmaceutical component, said different metabolic timings minimizing pharmacokinetic drug-drug interaction between said first and second pharmaceutical components when said first and second pharmaceutical components concurrently reside within the mammal.

39. The pharmaceutical combination for minimizing pharmacokinetic drug-drug interaction of claim 38, wherein the second pharmaceutical component is insoluble.

40. The pharmaceutical combination for minimizing pharmacokinetic drug-drug interaction of claim 39, wherein the second pharmaceutical component is administered with a drug delivery vehicle modification.

41. The pharmaceutical combination for minimizing pharmacokinetic drug-drug interaction of claim 40, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

42. The pharmaceutical combination for minimizing pharmacokinetic drug-drug interaction of claim 38, wherein the drug-metabolizing mechanism is an interaction with a particular species of drug-metabolizing enzymes.

43. The pharmaceutical combination for minimizing pharmacokinetic drug-drug interaction of claim 38, wherein the second pharmaceutical component is administered with a microemulsion drug delivery vehicle modification, wherein the pharmacokinetic profile of the second pharmaceutical component is altered by its association with the microemulsion.

44. The pharmaceutical combination for minimizing pharmacokinetic drug-drug interaction of claim 38, wherein the second pharmaceutical component is administered with an emulsion drug delivery vehicle modification, wherein the pharmacokinetic profile of the second pharmaceutical component is altered by its association with the emulsion.

45. The pharmaceutical combination for minimizing pharmacokinetic drug-drug interaction of claim 40, wherein the drug delivery vehicle modification further comprises surface modifiers and the pharmacokinetic profile of the second pharmaceutical component is altered by its association with surface modifiers.

46. The pharmaceutical combination for minimizing pharmacokinetic drug-drug interaction of claim 39, wherein the drug delivery vehicle modification is a nanosuspension of nanoparticles.

47. The pharmaceutical combination for minimizing pharmacokinetic drug-drug interaction of claim 37, wherein the second pharmaceutical component is itraconazole.

48. A method for minimizing pharmacokinetic drug-drug interaction in a mammal, comprising:
administering to the mammal a first pharmaceutical component that is metabolized by a particular drug-metabolizing mechanism according to a specific metabolic timing;
providing a second pharmaceutical component, the second component in a given formulation, when administered to the mammal, is metabolized by a similar drug-metabolizing mechanism and according to a similar metabolic timing as the first pharmaceutical component;
modifying the formulation of the second pharmaceutical component, wherein the modified formulation, when administered to the mammal, causes the second pharmaceutical component to be phagocytized in the MPS; and
administering the modified formulation of the second pharmaceutical component to the mammal parenterally, wherein phagocytosis of the modified formulation of the second pharmaceutical component results in a metabolic timing which is different from the metabolic timing of the second pharmaceutical component in the unmodified formulated state, said different metabolic timings minimizing pharmacokinetic drug-drug interaction between the first pharmaceutical component and the second pharmaceutical component when the first pharmaceutical component and the second pharmaceutical components concurrently reside within the mammal.

49. The method for minimizing drug-drug interaction in a mammal of claim 48, wherein the second pharmaceutical component is insoluble.

50. The method for minimizing drug-drug interaction in a mammal of claim 49, wherein the formulation of the second pharmaceutical component is modified via a drug delivery vehicle modification.

51. The method for minimizing drug-drug interaction in a mammal of claim 50, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

52. A method for minimizing pharmacokinetic drug-drug interaction in a mammal, comprising:
providing a first pharmaceutical component that is metabolized by a particular drug-metabolizing mechanism according to a specific metabolic timing;
providing a second pharmaceutical component, the second component in a given formulation, when administered to the mammal, is metabolized by a similar drug-metabolizing mechanism and according to a similar metabolic timing as the first pharmaceutical component;
modifying the formulation of the second pharmaceutical component, wherein the modified formulation, when administered to the mammal, causes the second pharmaceutical component to be phagocytized in the MPS;
administering the modified formulation of the second pharmaceutical component to the mammal parenterally; and
administering to the mammal the first pharmaceutical component, wherein phagocytosis of the modified formulation of the second pharmaceutical component results in a metabolic timing which is different from the metabolic timing of the second pharmaceutical component in the unmodified state, said different metabolic timings minimizing pharmacokinetic drug-drug interaction between the first pharmaceutical component and the second pharmaceutical components when the first and the second pharmaceutical components concurrently reside within the mammal.

53. The method for minimizing drug-drug interaction in a mammal of claim 52, wherein the second pharmaceutical component is insoluble.

54. The method for minimizing drug-drug interaction in a mammal of claim 53, wherein the formulation of the second pharmaceutical component is modified via a drug delivery vehicle modification.

55. The method for minimizing drug-drug interaction in a mammal of claim 54, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

56. A pharmaceutical combination for minimizing pharmacokinetic drug-drug interaction within a mammal, the pharmaceutical combination comprising:
a first pharmaceutical component selected from a group consisting of antiarrhythmics, anticonvulsants, antimycobacterials, antineoplastics, antipsychotics, benzodidiazepines, calcium channel blockers, gastrointestinal motility agents, HMG coAreductase inhibitors, immunosuppressants, oral hypoglycemics, protease inhibitors, levacetylmethadol, ergot alkaloids, halofantrins, alfentanil, buspirone, methylprednisolone, budesonide, dexamethsone, trimetrexate, warfarin, cilostazol, and cletripan, wherein said first pharmaceutical component has a particular pharmacokinetic profile in the mammal; and
a second pharmaceutical component of itraconazole formulated for parenteral administration, said second pharmaceutical component of itraconazole being formulated such that the pharmacokinetic profile of said second pharmaceutical component of itraconazole is altered from its unaltered pharmacokinetic profile, which unaltered profile substantially affects said particular pharmacokinetic profile of the first pharmaceutical component, so that said altered pharmacokinetic profile of said second pharmaceutical component of itraconazole does not substantially affect the pharmacokinetic profile of said first pharmaceutical component.

57. The pharmaceutical combination for minimizing drug-drug interaction in a mammal of claim 56, wherein said second pharmaceutical component of itraconazole is administered with a drug delivery vehicle modification.

58. The pharmaceutical combination for minimizing pharmacokinetic drug-drug interaction of claim 57, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.

59. A pharmaceutical combination for minimizing pharmacokinetic drug-drug interaction within a mammal, the pharmaceutical combination comprising:
a first pharmaceutical component of itraconazole in solution form, wherein said first pharmaceutical component of itraconazole has a particular pharmacokinetic profile in the mammal; and
a second pharmaceutical component selected from the group consisting of macrolide antibiotics and protease inhibitors formulated for parenteral administration said second pharmaceutical component being formulated such that the pharmacokinetic profile of said second pharmaceutical component is altered from its unaltered pharmacokinetic profile, which unaltered profile substantially affects said particular pharmacokinetic profile of the first pharmaceutical component of itraconazole, so that said altered pharmacokinetic profile of said second pharmaceutical component does not substantially affect the pharmacokinetic profile of said first pharmaceutical component of itraconazole.

60. The pharmaceutical combination for minimizing drug-drug interaction in a mammal of claim 59, wherein said second pharmaceutical component is administered with a drug delivery vehicle modification.

61. The pharmaceutical combination for minimizing pharmacokinetic drug-drug interaction of claim 60, wherein the drug delivery vehicle modification is selected from the group consisting of nanoparticles, salt formation, solid carrier systems, co-solvent/solubilization, micellization, lipid vesicle, oil-water partitioning, liposomes, microemulsions, emulsions, and complexation.
